(19) United States
(12) Patent Application Publication
(10) Pub. No.: US 2014/0255334 A1
(43) Pub. Date: Sep. 11, 2014

(54) BONE GRAFT MATERIALS CONTAINING CALCIUM PHOSPHATE AND POVIDONE-IODINE

(71) Applicant: ORTHOVITA, INC., Malvern, PA (US)

(72) Inventors: Jenny E. Raynor, Phoenixville, PA (US); Kristi L. Wagner, Coatesville, PA (US)

(21) Appl. No.: 13/788,943

(22) Filed: Mar. 7, 2013

(51) Int. Cl. A61K 31/79

(52) U.S. Cl. CPC A61K 31/79
USPC 424/78.25

(57) ABSTRACT

Described herein are materials and methods for reducing the risk of infection at a surgical site performed to restore or repair bone in an animal. Bone graft materials for implantation into a mammal are contemplated containing an antimicrobial agent. In one embodiment the bone graft material comprises both calcium phosphate and povidone-iodine as the antimicrobial agent. In another embodiment the bone graft material further comprises collagen. The bone graft materials are designed so as to maintain the structural integrity and/or handling characteristics of the material upon implantation into a bony site. Various methods for manufacturing the bone graft materials described herein are also contemplated.

### BACKGROUND OF THE INVENTION

Infection associated with surgery, including bone implant surgery, is a growing concern. Various materials have been tested for their use as an antimicrobial agent in surgical procedures. One such material is iodine. Solutions of iodine complexed with the polymer polyvinylpyrrolidone (herein after “povidone-iodine”) have been used to trap the iodine in order to provide a slow release of iodine at the surgical site, allowing for continued antimicrobial effect for an extended period of time. Such solutions of povidone-iodine have been shown to be effective in preventing infections from bacteria, fungi, protozoa, and mold. In addition, bacteria have not developed resistance to povidone-iodine, thus making it capable of killing methicillin-resistant Staphylococcus aureus (MRSA).

Povidone-iodine solutions are typically applied to the skin prior to surgery or immediately after closing an incision site in order to disinfect the area. Povidone-iodine solutions are also used in eye drops. David Bernard, “Povidone-Iodine Prevents Infection in Prosthetic Implants”, Outpatient Surgery E-Weekly (Mar. 8, 2011) teaches the effectiveness of soaking a surgical wound with a 10% solution of povidone-iodine after a total hip or knee procedure for the prevention of infection at the surgical site. However, the use of these povidone-iodine solutions requires the steps of applying the solution to the surgical site or washing of the wound with these antimicrobial solutions. In addition, the solution may not be retained at the surgical site for an extended period of time.

Accordingly, there is a need to incorporate an antimicrobial agent into a bone graft material in order to reduce the risk of infection at the surgical site, eliminate the need for additional washing/soaking steps, and retain the antimicrobial agent at the surgical site for an extended period of time after completion of the surgical procedure. Furthermore, the structural integrity of the bone implant must be maintained when incorporating the antimicrobial agent into the bone graft material.

### BRIEF SUMMARY OF THE INVENTION

Described herein are materials and methods for reducing the risk of infection at a surgical site performed to restore or repair bone in an animal. Bone graft materials for implantation into mammal comprising an antimicrobial agent are contemplated.

In one embodiment the bone graft material comprises both calcium phosphate and povidone-iodine as the antimicrobial agent. The mass ratio of povidone-iodine to calcium phosphate can be, for example, in the range of about 0.01:1 to 0.2:1. In another embodiment, the mass ratio of povidone-iodine to calcium phosphate is about 0.04:1 to about 0.15:1. In yet another embodiment, the mass ratio of povidone-iodine to calcium phosphate is at least about 0.08:1.

In another embodiment, the povidone-iodine leachate (amount of povidone-iodine that is released from the bone graft material) is at least about 4 milligrams when using a titration method described in Example 5. In another embodiment, the povidone-iodine leachate is at about 4 milligrams to about 100 milligrams in 1 hour when calculated using the titration method described in Example 5. In another embodiment, the povidone-iodine leachate is at about 4 milligrams to about 40 milligrams in 1 hour when calculated using the titration method described in Example 5.

In one embodiment, the povidone-iodine leachate (amount of povidone-iodine that is released from the bone graft material) is at least about 0.7% in 1 hour when calculated by the titration method described in Example 5. In another embodiment, the povidone-iodine leachate is about 0.7% to about 10% in 1 hour when calculated by the titration method described in Example 5. In yet another embodiment, the povidone-iodine leachate is about 0.7% to about 2% in 1 hour when calculated by the titration method described in Example 5.

In one embodiment, the biocompatible bone graft material comprises calcium phosphate, for example, O-tricalcium phosphate, containing macro-, meso-, and microporosity.

In another embodiment the bone graft material further comprises collagen. The bone graft materials are designed so as to maintain the structural integrity and/or handling characteristics of the material upon implantation into a bony site. The mass ratio of povidone-iodine to calcium phosphate when combined with collagen can be, for example, greater than about 0.04:1. In another example, the mass ratio of povidone-iodine to calcium phosphate when combined with collagen is about 0.15:1 to about 0.6:1.

In one embodiment, the ratio of the combination of calcium phosphate and povidone-iodine to collagen in the bone graft material is at least about 10 grams of calcium phosphate combined with povidone-iodine to about 1 gram of collagen. In another embodiment, the ratio of the combination of calcium phosphate and povidone-iodine to collagen in the bone graft material is at least about 5 grams of calcium phosphate combined with povidone-iodine to about 1 gram of collagen. In yet another embodiment, the ratio of the combination of calcium phosphate and povidone-iodine to collagen in the bone graft material is at least about 2 grams of calcium phosphate combined with povidone-iodine to about 1 gram of collagen.

In one embodiment, the collagen in the bone graft material is selected from the group consisting of non-crosslinked collagen pellet, lyophilized non-cross-linked collagen, and cross-linked collagen. Exemplary cross-linking agents include N-(3-dimethylaminopropyl-N′-ethylcarbodiimide hydrochloride and N-hydroxysuccinimde or glutaraldehyde.

In one embodiment, the biocompatible bone graft material comprises a homogenous blend of calcium phosphate and collagen.

Also contemplated are methods of using the various bone graft materials described herein for reducing the risk of infection while restoring or repairing bone in an animal comprising placing in the bone, at a site to be restored or repaired, the biocompatible bone graft materials described herein. In one embodiment, the bone graft material is wetted with a fluid prior to placing the bone graft material into the site to be restored or repaired. In another embodiment, the bone graft material is flexible, flowable, or moldable upon wetting.

Various methods for manufacturing the bone graft materials described herein are also contemplated. In one embodiment the method for preparing a biocompatible bone graft material comprising calcium phosphate and povidone-iodine includes preparing a solution of povidone-iodine; adding calcium phosphate to the solution to form a mixture; stirring the mixture; freezing the mixture; and lyophilizing the frozen mixture to form the biocompatible bone graft material comprising calcium phosphate and povidone-iodine.

In one embodiment, the solution of povidone-iodine is about 0.5% to about 10% weight by volume. In another embodiment, the solution of povidone-iodine is about 2.5% to about 10% weight by volume.

In certain embodiments, the mixture is stirred for at least about 1 hour prior to freezing the mixture. In another embodiment, the freezing of the mixture is performed at a temperature of about −28° C. to about 0° C.

In one embodiment a method for preparing a biocompatible bone graft material comprising calcium phosphate, povidone-iodine, and collagen includes preparing an solution of povidone-iodine; adding calcium phosphate to the solution to form a mixture; stirring the mixture; freezing the mixture; and lyophilizing the frozen mixture; mixing the lyophilized material with collagen to form a second mixture; freezing the second mixture; and lyophilizing the second mixture to form a biocompatible bone graft material comprising calcium phosphate, povidone-iodine, and collagen.

In one embodiment, the concentration of the solution of the povidone-iodine is at least about 5% weight by volume. In another embodiment, the concentration of the solution of the povidone-iodine is at least about 10% weight by volume.

In embodiment, the collagen is pre-mixed with povidone-iodine prior to mixing the collagen with the biocompatible bone graft material comprising calcium phosphate and povidone-iodine.

Additional methods for preparing a biocompatible bone graft material comprising calcium phosphate, collagen, and povidone-iodine include obtaining a bone graft material comprising calcium phosphate collagen; and soaking the bone graft material with a solution of povidone-iodine.

Also contemplated is a kit comprising a first component comprising a bone graft material comprising calcium phosphate and collagen; and a second component comprising a solution of povidone-iodine.

### BRIEF DESCRIPTION OF THE DRAWINGS

FIG. 1 demonstrates possible interactions between the amino acids of collagen and iodine-containing solutions.

FIG. 2 contains Scanning Electron Microscopy (SEM) images of various bone graft materials containing povidone-iodine and calcium phosphate.

FIG. 3 summarizes the results of Fourier-Transform Infrared Spectroscopy (FTIR) analysis on various bone graft materials containing povidone-iodine and calcium phosphate.

FIG. 4 summarizes the results of antimicrobial testing for various bone graft materials containing povidone-iodine and calcium phosphate.

FIG. 5 summarizes the results of Differential Scanning Calorimetry (DSC) on various collagen-containing bone graft materials.

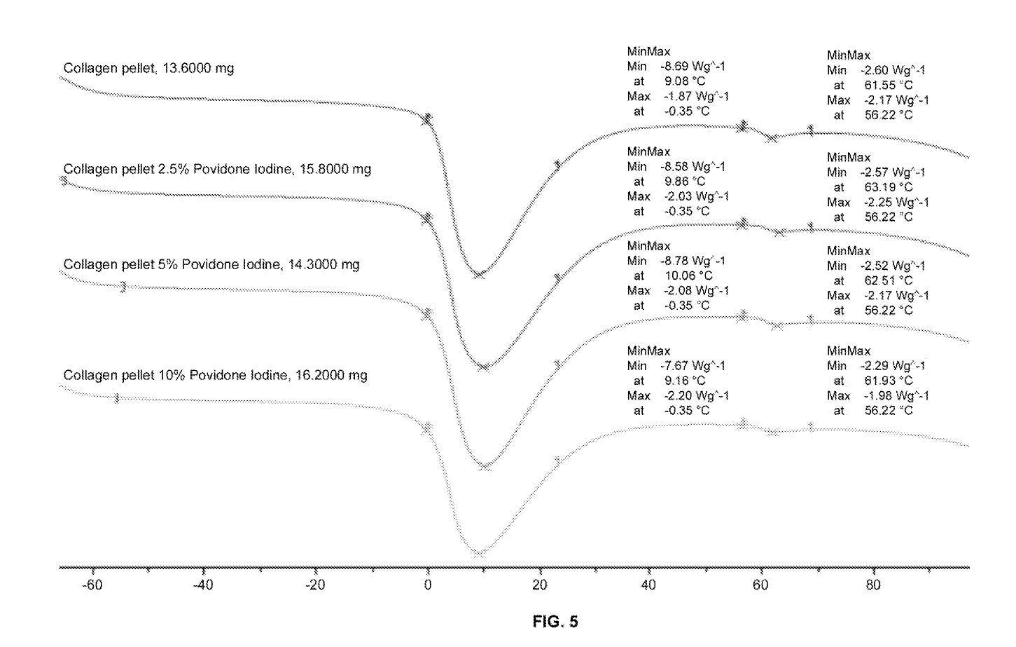

### DETAILED DESCRIPTION

The invention will be described in more detail below.

While the specification concludes with the claims particularly pointing out and distinctly claiming the invention, it is believed that the invention described herein will be better understood from the following description. All temperatures are in Degrees Celsius unless specified otherwise. The invention described herein can comprise (i.e., open ended) or consist essentially of the components of the invention described herein as well as other ingredients or elements described herein. As used herein, “comprising” means the elements recited, or their equivalent in structure or function, plus any other element or elements which are not recited. The terms “having,” “including,” and “comprised of” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Generally, such additives may not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value.

The term “leachate” used herein refers to the amount of iodine released from the bone graft material upon exposure to a fluid. A titration test is performed in order to calculate the leachate and is described in more detail below. The leachate can be expressed in milligrams of iodine or in percent of iodine in solution. The term “titration method” used herein refers to the methods described in Example 5.

### Bone Graft Materials Containing Calcium Phosphate and Iodine

Described herein are biocompatible bone graft materials for use in reducing the risk of infection at a surgical site performed to restore or repair bone in an animal. In one embodiment, the biocompatible bone graft materials described herein comprise a calcium salt and iodine.

Various calcium salts are contemplated and include, for example, calcium phosphates such as tricalcium phosphate, β-tricalcium phosphate (β-TCP) and α-tricalcium phosphate (α-TCP), and apatites such as hydroxyapatite. However, for the sake of brevity, “calcium phosphate” includes any calcium salt known to those skilled in the art. The preparation of various forms of calcium phosphate for use in the present invention is described in U.S. Pat. Nos. 6,383,519 and 6,521,246, assigned to the assignee of the present invention and incorporated herein by references in their entireties. An exemplary calcium phosphate product is Vitoss® Bone Graft Substitute (Orthovita, Inc., Malvern, Pa.).

The antimicrobial agent described herein can be any form of iodine, including, for example, iodine salts or iodine complexed with a polymer such as povidone or cadexomer.

In one embodiment, the bone graft material comprises calcium phosphate and povidone-iodine. The mass ratio of povidone-iodine (in grams) to calcium phosphate (in grams) is in the range of about 0.001:1 to about 0.2:1. In another embodiment, the mass ratio of povidone-iodine (in grams) to calcium phosphate (in grams) is in the range of about 0.04:1 to about 0.15:1. In yet another embodiment, the mass ratio of povidone-iodine (in grams) to calcium phosphate (in grams) is greater than about 0.08:1.

In one embodiment, the bone graft material comprises calcium phosphate and povidone-iodine such that the povidone-iodine leachate is at least about 4 milligrams in 1 hour when measured using the titration test described in Example 5. In another embodiment, the bone graft material comprises calcium phosphate and povidone-iodine such that the povidone-iodine leachate is at about 4 milligrams to about 100 milligrams in 1 hour when measured using the titration test described in Example 5. In yet another embodiment, the bone graft material comprises calcium phosphate and povidone-iodine such that the povidone-iodine leachate is at about 4 milligrams to about 40 milligrams in 1 hour when measured using the titration test described in Example 5.

Leachate can also be measured in terms of percent povidone-iodine in solution. In one embodiment, the bone graft material comprises calcium phosphate and povidone-iodine such that the povidone-iodine leachate is at least about 0.7% in 1 hour when measured using the titration test described in Example 5. In another embodiment, the bone graft material comprises calcium phosphate and povidone-iodine such that the povidone-iodine leachate is at about 0.7% to about 10% in 1 hour when measured using the titration test described in Example 5. In yet another embodiment, the bone graft material comprises calcium phosphate and povidone-iodine such that the povidone-iodine leachate is at about 0.7% to about 2% in 1 hour when measured using the titration test described in Example 5.

In one embodiment, the bone graft material comprises calcium phosphate and povidone-iodine but does not contain collagen.

In one embodiment the calcium phosphate is β-TCP. In typical embodiments the calcium phosphate is porous. In another embodiment, the calcium phosphate contains micro-, meso-, and macroporosity. In yet another embodiment the porosity of the calcium phosphate is interconnected. Macroporosity is characterized by pore diameters greater than about 100 μm and, in some embodiments, up to about 1000 μm to 2000 μm. Mesoporosity is characterized by pore diameters between about 100 μm and 10 μm, while microporosity occurs when pores have diameters below about 10 μm. It is preferred that macro-, meso-, and microporosity occur simultaneously and are interconnected in products of the invention. It is not necessary to quantify each type of porosity to a high degree. Rather, persons skilled in the art can easily determine whether a material has each type of porosity through examination, such as through the preferred methods of mercury intrusion porosimetry, helium pycnometry and scanning electron microscopy. While it is certainly true that more than one or a few pores within the requisite size range are needed in order to characterize a sample as having a substantial degree of that particular form of porosity, no specific number or percentage is called for. Rather, a qualitative evaluation by persons skilled in the art shall be used to determine macro-, meso-, and microporosity.

It will be appreciated that in some embodiments of materials prepared in accordance with this invention the overall porosity will be high. This characteristic is measured by pore volume, expressed as a percentage. Zero percent pore volume refers to a fully dense material, which, perforce, has no pores at all. One hundred percent pore volume cannot meaningfully exist since the same would refer to “all pores” or air. Persons skilled in the art understand the concept of pore volume, however and can easily calculate and apply it. For example, pore volume may be determined in accordance with Kingery, W. D., Introduction to Ceramics, Wiley Series on the Science and Technology of Materials, 1^{st} Ed., Hollowman, J. H., et al. (Eds.), Wiley & Sons, 1960, p. 409-417, who provides a formula for determination of porosity. Expressing porosity as a percentage yields pore volume. The formula is: Pore Volume=(1−fₚ) 100%, where fₚ is fraction of theoretical density achieved.

Porosity can be measured by Helium Pycnometry. This procedure determines the density and true volume of a sample by measuring the pressure change of helium in a calibrated volume. A sample of known weight and dimensions is placed in the pycnometer, which determines density and volume. From the sample's mass, the pycnometer determines true density and volume. From measured dimensions, apparent density and volume can be determined. Porosity of the sample is then calculated using (apparent volume-measured volume)/apparent volume. Porosity and pore size distribution may also be measured by mercury intrusion porosimetry.

Pore volumes in excess of about 30% may be achieved in accordance with this invention while materials having pore volumes in excess of 50% or 60% may also be routinely attainable. Some embodiments of the invention may have pore volumes of at least about 70%. Some embodiments that may be preferred have pore volumes in excess of about 75%, with 80% being still more preferred. Pore volumes greater than about 90% are possible as are volumes greater than about 92%. In some preferred cases, such high pore volumes are attained while also attaining the presence of macro-meso-, and microporosity as well as physical stability of the materials produced. It is believed to be a great advantage to prepare graft materials having macro-, meso-, and microporosity simultaneously with high pore volumes that also retain some compression resistance and flexibility when wetted.

In one embodiment, the bone graft material comprises porous calcium phosphate morsels at a size greater than about 0.25 mm. The morsels of calcium phosphate may be, for example, about 1-2 mm in size for some embodiments or about 0.25 mm to about 1 mm or to about 2 mm for other embodiments of the present invention. For flowable compositions, it will be appreciated that the morsel size will be selected considering the desired delivery apparatus. For example, for delivery of a flowable composition using a standard syringe, it will be necessary to select a morsel size that fits through the syringe orifice.

Due to the high porosity and broad pore size distribution (1 μm-1000 μm) of the present invention graft, the implant is not only able to wick/soak/imbibe materials very quickly, but is also capable of retaining them. A variety of fluids could be used with the present invention including blood, bone marrow aspirate, saline, antibiotics and proteins such as bone morphogenetic proteins (BMPs). Materials of the present invention can also be imbibed with cells (e.g., fibroblasts, mesenchymal, stromal, marrow and stem cells), platelet rich plasma, other biological fluids, and any combination of the above. Bone grafts of the present invention actually hold, maintain, and/or retain fluids once they are imbibed, allowing for contained, localized delivery of imbibed fluids. This capability has utility in cell-seeding, drug delivery, and delivery of biologic molecules as well as in the application of bone tissue engineering, orthopaedics, and carriers of pharmaceuticals.

Wettability determines the amount of fluid taken up by sample material and if the material absorbs an appropriate amount of fluid within a specified time. Pieces of the material are randomly selected, weighed, and placed in a container of fluid for 120 seconds. If the samples adequately take up fluid, they are then weighed again to determine the percentage of mass increase from fluid absorption. Some embodiments exhibit a wettability wherein bone graft material becomes fully saturated within 120 seconds with at least a 100% mass increase. In some embodiments, the graft material experiences a 150% mass increase and yet, in others, an approximate 200%-300% mass increase. Fluids that may be used in the present invention may be bone marrow aspirate, blood, saline, antibiotics and proteins such as bone morphogenetic proteins (BMPs) and the like.

It is preferred that flexible grafts of the present invention will be able to wick and hold fluids, even under compression. It is preferred that moldable embodiments will be able to wick and hold fluids, even in a wet environment. For example, if a wetted, flexible graft is placed on mesh suspended above a weigh boat and is challenged with a 500 g weight, it is preferred that the graft maintain a mass of fluid at least about 95% of the mass of the graft or about equivalent to the mass of the graft. If a wetted, moldable graft of the invention is placed in fluid, it is preferred that the graft maintains as a continuous object and does not swell substantially larger in size than its original dimensions. In some instances, the graft does not swell in size greater than about 50% more than its original dimensions, by qualitative assessment. If a wetted, moldable graft of the invention is compressed, it is preferred that the graft maintain a mass of fluid at least about 85% of the mass of the graft or about equivalent to the mass of the graft. Bone graft materials of the present invention have osteoconductive and osteostimulatory properties. In certain embodiments, the addition of bioactive glass in the present invention enhances the ability of the product to foster bone growth. The bone graft materials of the present invention may also have osteoinductive properties.

### Bone Graft Materials Containing Calcium Phosphate, Iodine, and Collagen

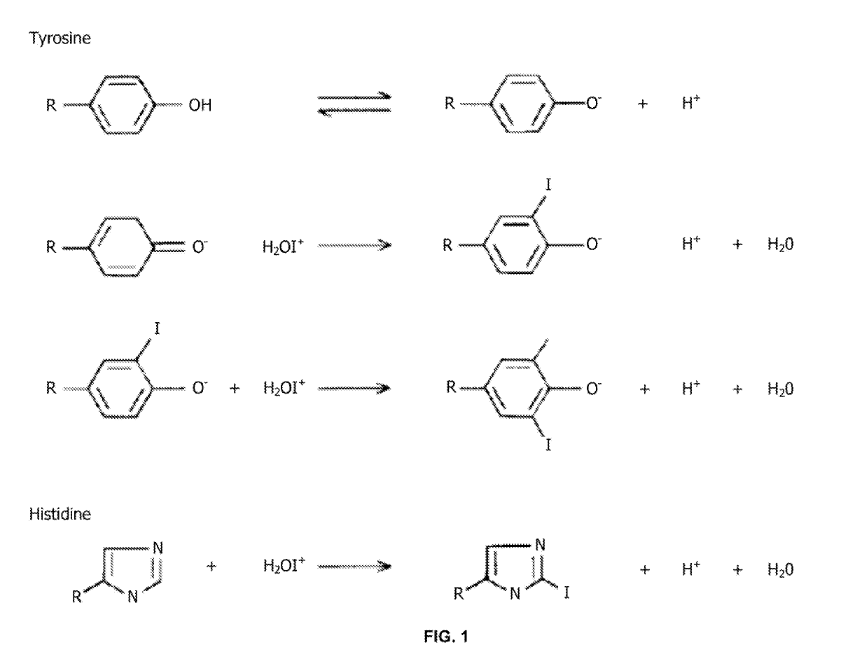

In one embodiment, the bone graft material contains calcium phosphate, povidone-iodine, and collagen. It has been discovered that iodine ions can interact with amino acid side chains on collagen thereby slowing the release of iodine from the bone graft material. Without being bound by a particular theory, it is believed that iodine may interact with tyrosine, tryptophan, and histidine residues of the collagen causing a delayed release of iodine from the scaffold. FIG. 1 demonstrates possible interactions between amino acids of collagen and iodine.

Accordingly, in one embodiment, an excess amount of povidone-iodine can be added to the bone graft material to ensure that after the amino acids of the collagen have complexed with some of the iodine, enough iodine will remain free to diffuse from the bone graft material and prevent infection at the bony site. The way to test to ensure that an effective amount of iodine can be released from the bone graft material is by performing the titration method described in Example 8.

In one embodiment, for a bone graft material comprising calcium phosphate, povidone-iodine, and collagen, wherein the mass ratio of povidone-iodine (in grams) to calcium phosphate (in grams) is greater than about 0.04:1. In another embodiment, for a bone graft material comprising calcium phosphate, povidone-iodine, and collagen, wherein the mass ratio of povidone-iodine (in grams) to calcium phosphate (in grams) is greater than about 0.15:1. In another embodiment, for a bone graft material comprising calcium phosphate, povidone-iodine, and collagen, wherein the mass ratio of povidone-iodine (in grams) to calcium phosphate (in grams) is about 0.15:1 to 0.6:1.

In one embodiment, the bone graft material comprises calcium phosphate, povidone-iodine, and collagen such that the povidone-iodine leachate is at least about 4 milligrams in 1 hour when measured using the titration test described in Example 5. In another embodiment, the bone graft material comprises calcium phosphate, povidone-iodine, and collagen such that the povidone-iodine leachate is at about 4 milligrams to about 100 milligrams in 1 hour when measured using the titration test described in Example 5. In yet another embodiment, the bone graft material comprises calcium phosphate, povidone-iodine, and collagen such that the povidone-iodine leachate is at about 4 milligrams to about 40 milligrams in 1 hour when measured using the titration test described in Example 5.

Leachate can also be measured in terms of percent povidone-iodine in solution. In one embodiment, the bone graft material comprises calcium phosphate, povidone-iodine, and collagen such that the povidone-iodine leachate is at least about 0.7% in 1 hour when measured using the titration test described in Example 5. In another embodiment, the bone graft material comprises calcium phosphate, povidone-iodine, and collagen such that the povidone-iodine leachate is at about 0.7% to about 10% in 1 hour when measured using the titration test described in Example 5. In yet another embodiment, the bone graft material comprises calcium phosphate, povidone-iodine, and collagen such that the povidone-iodine leachate is at about 0.7% to about 2% in 1 hour when measured using the titration test described in Example 5.

In one embodiment, the bone graft material comprises calcium phosphate, povidone-iodine, and collagen, wherein the mass ratio of the calcium phosphate and povidone-iodine to collagen is at least about 10 grams of calcium phosphate combined with povidone-iodine to about 1 gram of collagen. In another embodiment, the ratio of the combination of the calcium phosphate and povidone-iodine to collagen is at least about 5 grams of calcium phosphate combined with povidone-iodine to about 1 gram of collagen. In another embodiment, the ratio of the combination of the calcium phosphate and povidone-iodine to collagen is at least about 2 grams of calcium phosphate combined with povidone-iodine to about 1 gram of collagen.

Collagens suitable for use in the present invention may consist of non-crosslinked collagen pellet, lyophilized non-crosslinked collagen, and crosslinked collagen. The collagen may also be a telopeptide collagen, e.g., native collagen. Some embodiments of the present invention contain collagen that comprises up to 100% Type I collagen. In other embodiments, the collagens used may be predominantly, or up to about 90%, of Type I collagen with up to about 5% of Type III collagen or up to about 5% of other types of collagen. Suitable Type I collagens include native fibrous insoluble human, bovine, porcine, or synthetic collagen, soluble collagen, reconstituted collagen, or combinations thereof. Microfibrillar forms of collagen are also contemplated. Examples of suitable collagens are described, for example, in U.S. Pat. Nos. 6,096,309, 6,280,727, and 7,189,263, and U.S. Patent Publication Application No. 2011/0243913, which are herein incorporated by reference in their entirety.

In one embodiment the collagen is cross-linked with one selected from the group consisting of N-(3-dimethylaminopropyl-N′-ethylcarbodiimide hydrochloride and N-hydroxysuccinimde; and glutaraldehyde.

In one embodiment the biocompatible bone graft material comprises a homogenous blend of calcium phosphate and collagen.

The bone graft materials described herein may be flexible or moldable, or the materials may be flowable. The nature of the collagen affects the flexibility, moldability, or flowability of the graft material. A graft containing predominantly fibrous collagen will be flexible or moldable upon wetting, depending on the degree of cross-linking of the collagen. A graft containing primarily soluble collagen with limited or no cross-links will be flowable upon wetting.

### Methods of Using the Bone Graft Materials

The bone graft materials described herein may be used to prevent infection while restoring or repairing bone in an animal. These methods include placing in the bone, at a site to be restored or repaired, the biocompatible bone graft materials described herein. In one embodiment the bone graft material is wetted with a fluid prior to placement in the bony site. In another embodiment, the wetted bone graft material is flexible, moldable or flowable.

Many of the embodiments disclosed herein are to fill bony voids and defects. It will be appreciated that applications for the embodiments of the present invention include, but are not limited to, filling interbody fusion devices/cages (ring cages, cylindrical cages), placement adjacent to cages (i.e., in front cages), placement in the posterolateral gutters in posterolateral fusion (PLF) procedures, backfilling the iliac crest, acetabular reconstruction and revision hips and knees, large tumor voids, use in high tibial osteotomy, burr hole filling, and use in other cranial defects. The bone graft material strips may be suited for use in posterolateral fusion (PLF) by placement in the posterolateral gutters, and in onlay fusion grafting. Additional uses may include craniofacial and trauma procedures that require covering or wrapping of the injured/void site. The bone graft material cylinders may be suited to fill spinal cages and large bone voids, and for placement along the posterolateral gutters in the spine.

### Methods of Manufacturing Bone Graft Materials

Described herein are methods for manufacturing the biocompatible bone graft materials described herein. One exemplary embodiment for preparing a bone graft material comprising calcium phosphate and povidone-iodine includes preparing a solution of povidone-iodine; adding calcium phosphate to the solution to form a mixture; stirring the mixture; freezing the mixture; and lyophilizing the frozen mixture to form a biocompatible bone graft material comprising calcium phosphate and povidone-iodine.

In one embodiment, the concentration of the povidone-iodine solution is about 0.5% to about 10% weight by volume. In another embodiment, the concentration of the povidone-iodine solution is about 2.5% to about 10% weight by volume. In yet another embodiment, the concentration of the povidone-iodine solution is at least about 5% weight by volume.

In one embodiment the ratio of the calcium phosphate to the solution of povidone-iodine is about 0.065 grams of the calcium phosphate per about 1 milliliter of the povidone-iodine solution to about 6.5 grams of the calcium phosphate per about 1 milliliter of the povidone-iodine solution. In yet another embodiment, the ratio of the calcium phosphate to the solution of povidone-iodine is about 0.65 grams of the calcium phosphate per about 1 milliliter of the povidone-iodine solution.

In one embodiment, the mixture is stirred for at least about 1 hour. In another embodiment, the freezing of the mixture is performed at a temperature of about −28° C. to about 0° C. In yet another embodiment, the freezing of the mixture is performed at a temperature of about −28° C.

In one exemplary embodiment, methods for preparing a bone graft material comprising calcium phosphate, povidone-iodine, and collagen include preparing a solution of povidone-iodine; adding calcium phosphate to the solution to form a mixture; stirring the mixture; freezing the mixture; lyophilizing the frozen mixture; mixing the mixture of calcium phosphate and povidone-iodine with collagen to form a second mixture; freezing the second mixture; and lyophilizing the second mixture to form a biocompatible bone graft material comprising calcium phosphate, povidone-iodine, and collagen. In this embodiment, a cohesive mass of bone graft material is maintained with the addition of collagen.

In one embodiment, the concentration of the povidone-iodine solution is at least about 2.5% weight by volume. In another embodiment, the concentration of the povidone-iodine solution is at least about 10% weight by volume.

In another embodiment, methods for preparing a bone graft material comprising calcium phosphate, collagen, and povidone-iodine include soaking a bone graft material comprising calcium phosphate and collagen with a solution of povidone-iodine prior to placing into a bony site.

In another embodiment, a kit is contemplated that contains a first component comprising the bone graft material described herein containing calcium phosphate and collagen; and a second component comprising a solution of povidone iodine.

### EXAMPLES

### Example 1

### Preparation of Bone Graft Materials Containing Povidone-Iodine and Calcium Phosphate

Several different bone graft materials containing calcium phosphate and povidone-iodine were prepared with increasing amounts of povidone-iodine. Approximately 13.0 g of calcium phosphate particles were added to 20 ml of an aqueous povidone-iodine solution at concentrations of 0.5%, 1.0%, 2.5%, 5.0%, 7.5%, and 10.0% povidone-iodine. The mixtures were then frozen and lyophilized until dry. The resulting mass of povidone-iodine per gram of calcium phosphate is summarized in Table 1 below.

TABLE 1

| | | | Povidone- |
|---|---|---|---|
| | | | iodine per |
| Povidone- | Povidone- | Calcium | calcium |
| iodine | iodine | phosphate | phosphate |
| (%) | (mL) | (grams) | (grams/grams) |
| 0.5 | 20 | 13 | 0.007 |
| 1.0 | 20 | 13 | 0.0154 |
| 2.5 | 20 | 13 | 0.0386 |
| 5.0 | 20 | 13 | 0.0769 |
| 7.5 | 20 | 13 | 0.1154 |
| 10.0 | 20 | 13 | 0.1538 |

### Example 2

### Structure and Handling Characteristics of Bone Graft Materials

At lower concentrations of povidone-iodine the bone graft material was brittle and easily broke into small granules. However, as the concentration of povidone-iodine increased, the bone graft material became less brittle. With higher concentrations of povidone-iodine the povidone-iodine solution can act like a gel, holding together the calcium phosphate to form larger particles.

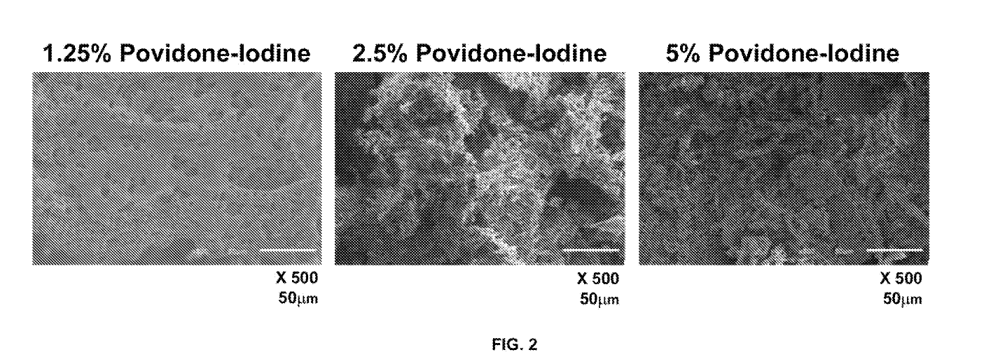

In order to determine whether the addition of povidone-iodine had any effect on the structure of the calcium phosphate particles, Scanning Electron Microscopy (SEM) images were obtained for the scaffold prepared with a 1.25%, 2.5% and 5.0% povidone-iodine solution. The SEM images at 500× magnification are reproduced as FIG. 2 and demonstrate that the bone graft material has an irregular, highly porous structure. In addition, the overall structure of the bone graft material does not change with increasing amounts of povidone-iodine. This highly porous structure allows for nutrient transport as cells infiltrate the calcium phosphate after implantation into a bony site. Furthermore, there was an increase in particle size of the bone graft material as the concentration of povidone-iodine solution increased. This increase in particle size indicates that increasing amounts of povidone-iodine solution produce a material that is capable of maintaining the structural integrity of the resulting bone graft material.

### Example 3

### Fourier-Transform Infrared Spectroscopy

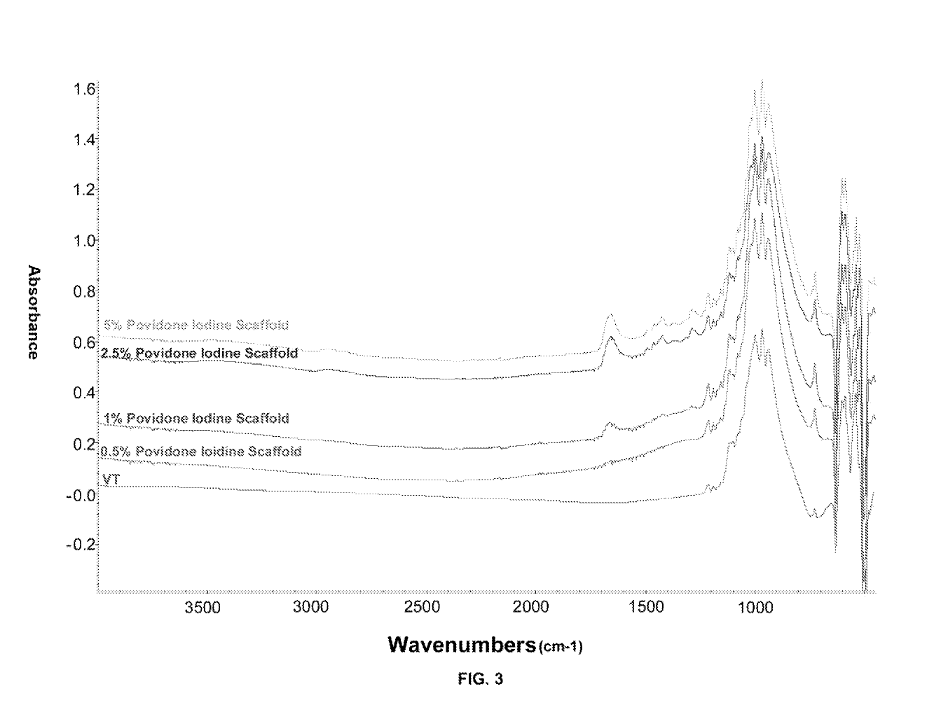

Fourier-Transform Infrared Spectroscopy (FTIR) was used to analyze the samples prepared above in order to determine if any chemical changes occur to the calcium phosphate upon preparation of the bone graft material. As a control, unmodified calcium phosphate was also tested. The FTIR spectra results are summarized in FIG. 3 and show two distinct new absorbance peaks to the calcium phosphate spectra that can be attributed to the addition of povidone-iodine as the intensity of these peaks decreases with decreasing amounts of povidone-iodine. More importantly there is no significant change to the spectrum of unmodified calcium phosphate, which indicates that the povidone-iodine does not degrade or alter the structure of the calcium phosphate particles upon preparation of the bone graft material.

### Example 4

### Antimicrobial Testing

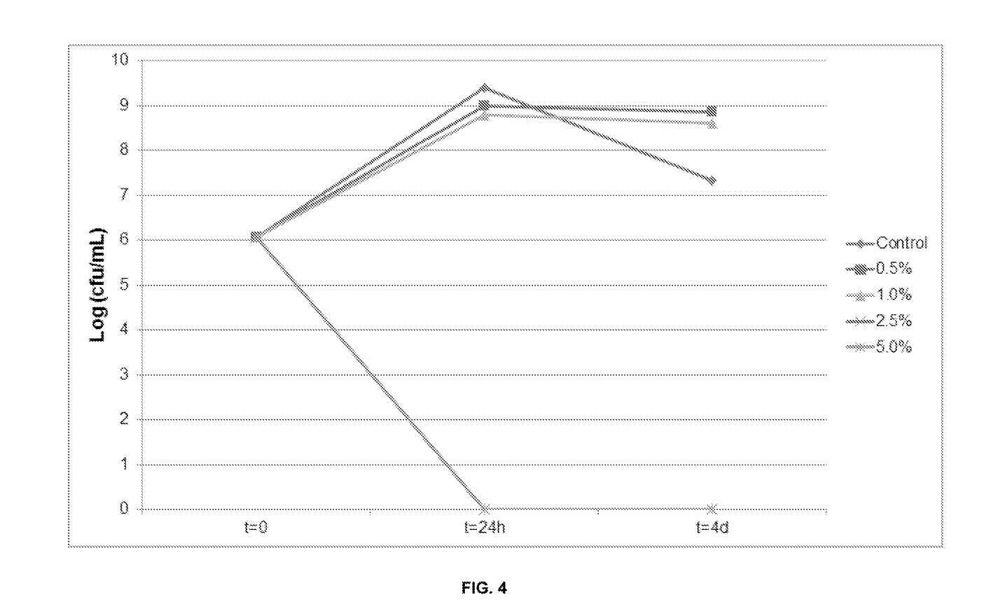

Povidone-iodine/calcium phosphate bone graft materials were prepared with either a 0.5%, 1.0%, 2.5%, or 5.0% povidone-iodine as described in Example 1. The bone graft materials were added to Tryptic Soy Broth (TSB) at a ratio of 0.2 g of scaffold per ml of broth. Staphylococcus aureus was added to the scaffold/broth mixture at a target concentration of 1×10⁶ cfu/ml and incubated at 37° C. for up to 4 days. An aliquot of each sample were taken at 0 hour, 24 hour and 4 days, serially diluted, plated, onto agar plates. Bacterial colonies were counted after 24 hours. FIG. 4, which summarizes the results of the plate count, demonstrates that bone graft materials made with a concentration of povidone-iodine solution of greater than 1.0% provide antimicrobial properties.

### Example 5

### Determination of Iodine Leachate

Scaffolds prepared with 2.5%, 5.0%, and 10% povidone-iodine solution as described in Example 1 were used to determine the amount of iodine released from the scaffold after contact with a solution. The amount of iodine released from each of the scaffolds was determined after the scaffold was in contact with water for 1 hour. The amount of iodine released into the water after 1 hour was determined by using the titration method described by Ohta et al., Biol. Pharm. Bull., Vol. 1 pp. 42-47 (1999), incorporated by reference herein in its entirety. Briefly, approximately 2.5 g of the bone graft material was incubated in 12.5 mL USP water for 1 hour. After incubation 6.25 mL of the supernatant was transferred to a clean, dry flask and 0.5 mL of a 0.5% starch solution was added. The iodine and starch solution was dark brown or black and color. The iodine and starch solution was titrated using a 0.005 M sodium thiosulfate solution until the solution became clear. During the course of the reaction the following color changes should be observed: the povidone iodine solution should turn black upon addition of starch, the solution should change from black to green to blue and finally become clear when the end point of the titration is reached. The mass of iodine in solution was calculated based on the volume of sodium thiosulfate required to make the iodine containing solution clear. The results in Table 2 below summarize the calculated amount of iodine in solution based on the titration method and the fact that each 1.0 mL of 1.0 M sodium thiosulfate used correlates to 12.69 mg of iodine. As a control, solutions with known concentrations of povidone-iodine at 2.5% and 5.0% were also tested.

TABLE 2

| | | | Theoretical | |
|---|---|---|---|---|
| | | Calculated | Mass of | Concentration |
| | Volume of | Iodine in | Iodine in | of Povidone |
| | Na₂S₂O₃ | Solution | Scaffold | Iodine |
| Sample | (mL) | (mg) | (mg) | (%) |
| Material made | 6.7 | 4.25 | 3.58 | 0.7 |
| with 2.5% | | | | |
| povidone- | | | | |
| iodine | | | | |
| Material made | 11.8 | 7.49 | 7.16 | 1.24 |
| with 5% | | | | |
| povidone- | | | | |
| iodine | | | | |
| Material made | 12 | 7.61 | 7.16 | 1.26 |
| with 5% | | | | |
| povidone- | | | | |
| iodine | | | | |
| Control - 2.5% | 26 | 16.5 | 15.12 | 2.73 |
| povidone- | | | | |
| iodine | | | | |
| solution | | | | |
| Control 5% | 51.5 | 32.68 | 30.24 | 5.4 |
| povidone- | | | | |
| iodine | | | | |
| solution | | | | |

The results indicate that most, if not all, of the iodine was released from the scaffold within 1 hour of exposure to a solution of water.

This experiment was repeated with duplicate samples. One of the duplicate samples was sterilized by gamma sterilization at 25-40 kGy, while the other duplicate was not exposed to radiation. The results were nearly identical between the sterilized and non-sterilized bone graft materials indicating that the bone graft material is not chemically altered or degraded during the sterilization process (data not shown).

### Example 6

### Preparation of Bone Graft Materials Containing Collagen

Bone graft materials containing povidone-iodine, calcium phosphate, and collagen were prepared by first making a bone graft material of either 2.5% or 5.0% povidone-iodine and calcium phosphate as described in Example 1. Various types of collagen were added to the povidone-iodine/calcium phosphate materials including non-crosslinked collagen, freshly precipitated collagen pellet, lyophilized non-crosslinked collagen, and collagen crosslinked with either N-(3-dimethylaminopropyl)-N′-ethylcarbodiimide hydrochloride (EDC) and N-hydroxysuccinimide (NHS) or glutaraldehyde (GTA). To make the bone graft materials with collagen, the povidone-iodine/calcium phosphate material was added to each of the different types of collagen at a mass ratio of either 2 parts povidone-iodine/calcium phosphate scaffold to 1 part collagen or 1 part povidone-iodine/calcium phosphate scaffold to 1 part collagen. The mixtures were then frozen and lyophilized to dryness.

As discussed above, some of the bone graft materials containing povidone-iodine and calcium phosphate were brittle and granular in nature. However, upon mixture with collagen, the bone graft material became a cohesive mass.

### Example 7

### Antimicrobial Testing

Bone graft materials containing povidone-iodine, calcium phosphate, and collagen were prepared with a 2.5% povidone-iodine solution by first preparing a material containing povidone-iodine and calcium phosphate as described in Example 1 The povidone-iodine/calcium phosphate material was combined with various types of collagen including non-crosslinked collagen, freshly precipitated collagen pellet, lyophilized non-crosslinked collagen, and collagen crosslinked with either N-(3-dimethylaminopropyl)-N′-ethylcarbodiimide hydrochloride (EDC) and N-hydroxysuccinimide (NHS) or glutaraldehyde (GTA) crosslinked collagen. The mass ratio of povidone-iodine/calcium phosphate scaffold to collagen was either 2:1 or 1:1 (in grams per gram). The mixtures were then frozen and lyophilized until dry.

The collagen-containing bone graft materials were tested for antimicrobial properties as described in Example 4. No antibacterial properties were observed for any of these collagen-containing scaffolds.

### Example 8

### Determination of Iodine Leachate

Determination of the amount of iodine that is released from the various collagen-containing scaffolds was performed using the titration method discussed above and bone graft materials prepared with either a 5.0% povidone-iodine solution and non-crosslinked collagen at a ratio of 2:1, or a 5.0% povidone-iodine solution and cross-linked collagen at a ratio of 2:1. Calculation of the amount of released iodine was performed in the same manner as in Example 5. The results are summarized in Table 3 below.

TABLE 3

| | | | Theoretical | |
|---|---|---|---|---|
| | | Calculated | Mass of | Concentration |
| | Volume of | Iodine in | Iodine in | of Povidone |
| | Na₂S₂O₃ | Solution | Scaffold | Iodine |
| Sample | (mL) | (mg) | (mg) | (%) |
| Material made | 0 | 0 | 0 | 0 |
| with 2.5% | | | | |
| povidone-iodine | | | | |
| Material made | 0.45 | 0.29 | 4.77 | 0.12 |
| with 5% | | | | |
| povidone-iodine | | | | |
| and non- | | | | |
| crosslinked | | | | |
| collagen | | | | |
| Material made | 0.1 | 0.06 | 2.13 | 0.03 |
| with 5% | | | | |
| povidone-iodine | | | | |
| and crosslinked | | | | |
| collagen | | | | |

The results indicate that very little iodine is released from the collagen-containing scaffolds. Without being bound by a particular theory, it is believed that iodine can react with certain amino acid groups in the collagen to form a covalent bond. This bond formation may trap the iodine in the scaffold and prevents it from diffusing into solution. Therefore, a larger than expected amount, e.g., greater than 5% amount of a povidone-iodine solution, is needed to prepare bone graft materials containing collagen in order to ensure that iodine can be reproducibly and controllably released from the scaffold in a quantity sufficient to prevent infection; that is so the amount of leachate measured by the titration method as disclosed in Example 5 is within the effective range of at least 4 milligrams or at least 0.7%.

### Example 9

### Differential Scanning Calorimetry

The temperature at which the collagen denatures was determined after combination of collagen with a solution of povidone-iodine using Differential Scanning Calorimetry (DSC). Samples for testing by DSC were prepared by adding aqueous solutions of povidone-iodine, at concentrations of 2.5%, 5.0%, and 10.0%, to a collagen pellet. As a control, DSC analysis of a collagen pellet without incubation with a povidone-iodine solution was also performed. The results are summarized in the graph of FIG. 4. The results indicate that the addition of povidone-iodine to collagen did not change the melting temperature of the collagen at any concentration of povidone-iodine solution. Therefore, although the povidone-iodine may be covalently attaching to the collagen, this attachment does not does not alter or denature the collagen in any significant way.

### Example 10

### Addition of Povidone-Iodine Solution to Vitoss® Pack

Several solutions of povidone-iodine were made at concentrations of 2.5%, 5%, and 10% weight by volume. The solutions were added to bone graft materials containing calcium phosphate and collagen (Vitoss® Foam Bone Graft Substitute, Orthovita, Inc., Malvern, Pa.) until a permanent color change to brown was observed. Without being bound by theory, it is believed that a color change of the bone graft materials containing collagen from a white color to an orange-brown color upon addition of the povidone-iodine solution indicates that there is a sufficient amount of povidone-iodine solution that is not interacting with the amino acids of the collagen, and therefore, are free to be released from the bone graft material. The results are summarized in Table 4 below and indicate the amount of volume of povidone-iodine required to observe a permanent color change to orange-brown color in the bone graft material.

TABLE 4

| Povidone Iodine | Volume Povidone | Povidone Iodine |
|---|---|---|
| Concentration | Solution (mL) | (g) |
| 2.5 | 2.2 | 0.055 |
| 5.0 | 1.6 | 0.080 |
| 10.0 | 0.8 | 0.080 |

The results indicate that approximately at least 0.055 grams of povidone-iodine are required to be added to bone graft materials containing collagen in order to have enough povidone-iodine that is free to be released from the bone graft material, and thus maintain its antimicrobial properties.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A biocompatible bone graft material comprising calcium phosphate and povidone-iodine, wherein the mass ratio of povidone-iodine to calcium phosphate is about 0.001:1 to about 0.2:1.

2. The biocompatible bone graft material of claim 1, wherein the mass ratio of povidone-iodine to calcium phosphate is about 0.04:1 to about 0.15:1.

3. The biocompatible bone graft material of claim 1, wherein a povidone-iodine leachate is at least about 4 milligrams in 1 hour when calculated using the titration method.

4. The biocompatible bone graft material of claim 1, wherein a povidone-iodine leachate is at about 4 milligrams to about 100 milligrams in 1 hour when calculated using the titration method.

5. The biocompatible bone graft material of claim 1, wherein a povidone-iodine leachate is at least about 0.7% in 1 hour when calculated by the titration method.

6. The biocompatible bone graft material of claim 1, wherein a povidone-iodine leachate is about 0.7% to 10% in 1 hour when calculated by the titration method.

7. The biocompatible bone graft material of claim 1, further comprising collagen.

8. The biocompatible bone graft material of claim 7, wherein the mass ratio of povidone-iodine to calcium phosphate is greater than about 0.04:1.

9. The biocompatible bone graft material of claim 7, wherein the mass ratio of povidone-iodine to calcium phosphate is greater than about 0.15:1.

10. The biocompatible bone graft material of claim 7, wherein the ratio of the combination of calcium phosphate and povidone-iodine to collagen is at least about 10 grams of calcium phosphate combined with povidone-iodine to about 1 gram of collagen.

11. The biocompatible bone graft material of claim 7, wherein the ratio of the combination of calcium phosphate and povidone-iodine to collagen is at least about 5 grams of calcium phosphate combined with povidone-iodine to about 1 gram of collagen.

12. The biocompatible bone graft material of claim 7, wherein a povidone-iodine leachate is at least about 4 milligrams in 1 hour when calculated using the titration method.

13. The biocompatible bone graft material of claim 7, wherein a povidone-iodine leachate is at about 4 milligrams to about 100 milligrams in 1 hour when calculated using the titration method.

14. The biocompatible bone graft material of claim 7, wherein a povidone-iodine leachate is at least about 0.7% in 1 hour when calculated by the titration method.

15. The biocompatible bone graft material of claim 7, wherein a povidone-iodine leachate is about 0.7% to 10% in 1 hour when calculated by the titration method.

16. A method for reducing the risk of infection while restoring or repairing bone in an animal comprising placing in the bone, at a site to be restored or repaired, the biocompatible bone graft material of claim 1.

17. A method for preparing a biocompatible bone graft material comprising calcium phosphate and povidone-iodine comprising:
preparing a solution of povidone-iodine;
adding calcium phosphate to the solution to form a mixture;
stirring the mixture;
freezing the mixture; and
lyophilizing the frozen mixture to form the biocompatible bone graft material comprising calcium phosphate and povidone-iodine.

18. The method of claim 17, wherein the solution of povidone-iodine is about 0.5% to about 10% weight by volume.

19. The method of claim 17, wherein the solution of povidone-iodine is about 2.5% to about 10% weight by volume.

20. The method of claim 17 further comprising:
mixing the biocompatible bone graft material comprising calcium phosphate and povidone-iodine with collagen to form a second mixture;
freezing the second mixture; and
lyophilizing the second mixture to form a biocompatible bone graft material comprising calcium phosphate, povidone-iodine, and collagen.

21. The method of claim 20, wherein the concentration of povidone-iodine solution is at least about 5% weight by volume.

22. The method of claim 20, wherein the concentration of the aqueous solution of the povidone-iodine is at least about 10% weight by volume.

23. A method for preparing a biocompatible bone graft material comprising calcium phosphate, collagen, and povidone-iodine comprising:
obtaining a bone graft material comprising calcium phosphate collagen;
soaking the bone graft material with a solution of povidone-iodine.

24. A kit comprising
a first component comprising a bone graft material comprising calcium phosphate and collagen; and
a second component comprising a solution of povidone-iodine.
